(19) United States
(12) Patent Application Publication
(10) Pub. No.: US 2012/0107282 A1
(43) Pub. Date: May 3, 2012

(54) NEURAL STEM CELL COMPOSITION CAPABLE OF TREATING CANCER AND METHOD OF TREATMENT

(75) Inventor: Seung U. Kim, Suwon-si (KR)

(73) Assignee: S. BIOMEDICS CO., LTD., Seoul (KR)

(21) Appl. No.: 13/247,567

(22) Filed: Sep. 28, 2011

Related U.S. Application Data
(60) Provisional application No. 61/389,026, filed on Oct. 1, 2010.

(51) Int. Cl. A61K 35/12; A61P 35/00; A61K 31/513

(52) U.S. Cl. 424/93.7; 514/274

(57) ABSTRACT

Disclosed is a method of treatment of an individual suffering from primary brain tumors (glioma and medulloblastoma) and brain metastases of extracranial cancers using human stem cells encoding therapeutic genes. The method includes giving the individual a clinically acceptable therapeutic reagent by intravascular injection of a pharmaceutical composition. The pharmaceutical composition includes neural stem cells (NSCs) genetically engineered to express a suicide gene (cytosine deaminase) and a cytokine gene (IFN-β) and a pharmaceutical carrier suitable for injection. The NSCs migrate selectively to tumor site in the brain, target tumor cells, kill tumor cells, inhibit tumor growth and thus treat the tumor.

### CROSS REFERENCE TO RELATED APPLICATION(S)

This application claims the benefit of U.S. Provisional Patent Application Ser. No. 61/389,026, filed Oct. 1, 2010, the contents of which are incorporated by reference herein in their entirety.

### BACKGROUND OF THE INVENTION

1. Field of the Invention

The present invention relates to the field of cellular and molecular therapy with genetically modified and unmodified stem cells. Most particularly, the invention relates to a method of systemic treatment of central nervous system (CNS) tumors and other tumors in both intracranial/intraspinal and extracranial/extraspinal sites, using neural stem cells (NSCs).

2. Description of the Related Art

Malignant glioma represents about 20% of all intracranial tumors. Despite advances in radiation therapy and chemotherapy administered after the surgical resection, the prognosis of malignant glioma remains poor with a median survival of 10 months (Cancer 1993; 71:2585-2597). The infiltrative nature of malignant gliomas and the limited penetration of chemotherapeutic agents through the tight blood-brain barrier are obstacles in the treatment of these formidable tumors.

Genetically modified human neural stem cells (NSCs) selectively migrate toward brain tumor cells and deliver therapeutic agents with significant treatment effects. Human NSCs that are retrovirally transduced with suicide gene cytosine deaminase (CD) gene show a remarkable ‘bystander killer effect’ on brain tumor cells following administration of 5-fluorocytosine (5-FC) (Proc Natl Acad Sci USA 2000; 97:12846-12851; Clin Cancer Res 2006; 12:5550-5556; Gene Ther 2007; 14:1132-1142).

Interferon-β (IFN-β) is known for its ability to interfere with viral replication and also for its anti-proliferative effects on a variety of cancer cells. However, the efficacy of IFN-β is limited because of its extremely short half-life after intravenous administration as well as the systemic toxicity when this protein is administered at doses required to achieve the desired antitumor effect (Hum Gene Ther 2004; 15:77-86).

Research has shown that because of their remarkable migratory and tumor-tropic properties, human NSCs represent a potentially powerful tool for the treatment of brain tumors by delivering therapeutic drugs into the intracranial glioma across the blood-brain barrier (Nat Med 2000; 6:447-450; Cancer Gene Ther 2003; 10:396-402; Gene Ther 2007: 14:1132-1142).

Genetically modified human NSCs expressing both CD and IFN-β migrate into the intracranial tumor bed through the blood vessels, exhibit antitumor effect by the combined delivery of a suicide gene and a cytotoxic cytokine gene onto the glioma tumor and kill the tumor cells.

### SUMMARY OF THE INVENTION

The present invention is based upon a surprising finding that stem cells such as neural stem cells (NSCs), can migrate through the brain, track invading and/or metastatic tumor cells and, when administered locally in the brain or systemically via an intravascular route, cross the blood brain barrier to teach tumor cells in the brain. Stem cells administered into the cerebrospinal fluid (CSF) via intracisternal, intrathecal, or intraventricular routes can similarly enter the brain/spinal cord parenchyma. Genetically modified NSCs are found to target tumor cells, including metastatic tumor cells when delivered through the peripheral vasculature.

The present invention provides a method to treat tumors by administering modified NSCs to an individual bearing tumors.

The present invention provides a method for not only attacking the tumor proper but also for attacking and killing metastasizing tumor cells while minimizing harm to surrounding tissue.

In one embodiment, the present invention provides a method of treating a tumor in an individual in need thereof comprising providing modified NSCs encoding cytosine deaminase gene and interferon-β which are capable of migrating to the tumors (both in and outside the nervous system) and exerting a therapeutic effect and delivering two therapeutic agents available to the tumor cells.

The NSCs are genetically engineered to contain suicide gene. The suicide genes include cytosine deaminase and carboxylesterase and herpes simplex-1 thymidine kinase. The brain tumor and metastatic cancer are found in locations of, intracranial cavity, spinal cord or extracranial location. The brain tumor is glioma, medulloblastoma or metastatic cancer. Human NSCs are derived from a human fetal telencephalon and immortalized by v-myc gene. Inhibition of the tumor growth is caused by bystander killing effect by suicide gene-prodrug combination and interferon-β tumorcidal effect.

In a further embodiment of the present invention, NSC compositions capable of treating brain tumors and metastatic cancers in the brain by migrating through blood brain barrier include human NSCs genetically modified to encode suicide genes, IFN-β gene, and pharmaceutical carriers suitable for intravascular injection. The NSCs are genetically engineered to encode a suicide gene that includes cytosine deaminase, carboxylesterase or herpes simplex-1 thymidine kinase. The suicide gene is genetically engineered to express cytosine deaminase, carboxylesterase or herpes simplex-1 thymidine kinase by way of retroviral mediated transduction of a suicide gene. The NSCs are genetically engineered to express IFN-β, sTRAIL, IL-4 or IL-12 by way of retroviral mediated transduction of a cytokine that includes IFN-β, sTRAIL, IL-4 or IL-12. The NSCs and cytokine genes are, for example, human. Cytosine deaminase gene is, for example, E. Coli or yeast origin.

These and other features and advantages of the present invention will become apparent from the following detailed description of illustrated embodiments thereof, which is to be read in connection with the accompanying drawings

### BRIEF DESCRIPTION OF THE DRAWINGS

The above and other objects, features and other advantages of the present invention will be more clearly understood from the following detailed description when taken in conjunction with the accompanying drawings in which:

FIG. 1 shows the results of the reverse transcription PCR for three cell lines (F3 human NSCs, F3.CD human NSC expressing CD, and F3.CD.IFN-βhuman NSCs expressing both CD and IFN-β. The IFN-β transcript was expressed only in F3.CD.IFN-β human NSCs;

FIG. 2 shows a bystander killing effect by the human NSCs' The U251 human glioma cells were seeded in a 24-well plate and co-cultured with either F3.CD or F3.CD.IFN-β cells at various U251/F3 ratios ranging from 1:0 to 1:4. After the day 1 of culture, 5-fluorocytosine (5-FC) was added to the medium at a final concentration of 500 mg/mL, and the culture was maintained for 3 more days. Each experiment was performed in triplicate. The number of viable cells was counted by the trypan blue method, and expressed as the percentage of untreated U251 glioma cells. The number of viable cells decreased with an increase in the F3/U251 ratio in both the cocultures, that is, in F3.CD+U251 and F3.CD.IFN-β+U251. Notably, the bystander killing effect exhibited by the F3.CD.IFN-b cells was more significant than that of the F3.CD cells, at the F3/U251 ratio of 2:1 and 4:1 (*P<0.05);

FIG. 3 shows the results of clonogenic assay. U251 human glioma cells were seeded in a 24-well plate and co-cultured with either F3.CD or F3.CD.IFN-β cells at various U251/F3 ratios ranging from 1:0 to 1:4. After 24 h culture, 5-fluorocytosine (5-FC) was added to the medium at a final concentration of 500 mg/mL, and the culture was maintained for 3 more days. The surviving cells were detached from the plates and re-seeded in a six-well plate at a cell density of 500 cells per well (duplicate). The cells were cultured for 9 days and then stained with 0.25% 1,9-dimethylmethylene blue in 50% Ethanol. The number of colonies was counted by two independent observers. The clonogenic potential of surviving U251 glioma cells after co-culture with F3.CD.IFN-β after 5-FC treatment was significantly reduced compared with co-culture with F3.CD (*Po0.05);

FIG. 4 shows Migration assay in vitro. U251 human glioma cells were plated on the 24-well plate and cultured for 48 hr. CM-Dil-labeled F3 or F3.CD.IFN-β cells were seeded into the upper wells of the FluoroBlok inserts. After 24-hr incubation, migrated (bottom) and non-migrated cells (top) were counted. There was no significant difference between the migration indices of these two cell lines;

FIG. 5 shows Migration of F3.CD.IFN-β cells into the tumor parenchyma. To ascertain the migratory capability of the NSCs from the vessels to the tumor parenchyma, the Dil-labeled F3.CD. IFN-β cells (red) were injected into the tail vein, and the newly formed tumor vessels were immunostained by using an anti-CD34 antibody (green). The Dil-labeled NSCs were found to be present in both, tumor stroma and tumor parenchyma. An adjacent section was also stained with hematoxylin and eosin, and the field relevant to the fluorescence images is displayed. The arrows indicate tumor vessels (Scale bar, 100 μm);

FIG. 6 shows the growth inhibitory effect of genetically engineered NSCs on the glioma cells in vivo. The mice with the U251-derived intracerebral tumor were injected with F3 cells, and then i.p. injections of 5-fluorocytosine (5-FC) were administered for the next 10 days. The volume of the tumor was assessed on day 28 after FC treatment. The residual tumor mass obtained in the group treated with F3.CD. IFN-β cells was much smaller than that obtained in the group treated with F3.CD cells (*P<0.05); and

FIG. 7 is a chart demonstrating the survival time of experimental animals. Mice were inoculated with U251 human glioma cells intracranially, and subsequently with each type of human NSCs [F3.CD.IFN-b (n=8), F3.CD (n=6) and F3 (n=5)], followed by intraperitoneal injection of prodrug 5-fluorocytosine (5-FC) and compared with untreated animals (n=5).

We measured the survival time from U251 cell inoculation. The rates of survival of mice treated with F3.CD.IFN-b cells were significantly higher than those of mice treated with F3.CD (*P<0.05); mice that received F3.CD therapy had significantly higher survival rates than those that received only F3 cells (**P<0.005)

### DETAILED DESCRIPTION OF THE INVENTION

It should be understood that the exemplary embodiments of the present invention described below may be modified in different ways without departing from the inventive principles disclosed herein, and the scope of the present invention is therefore not limited to these particular following embodiments. Rather, these embodiments are provided so that this disclosure will be through and complete, and will fully convey the concept of the invention to those skilled in the art by way of example and not of limitation.

In an earlier pilot clinical trial, liposome-mediated IFN-β gene therapy has shown potent antitumor activity in patients with malignant glioma. Hum Gene Ther 2004; 15: 77-86; J GeneMed 2008; 10: 329-339. This clinical trial suggested that the IFN-β gene delivery permitted locally sustained IFN-β production at levels sufficient to yield antitumor efficacy with minimal systemic adverse effects.

In the present invention, intravenously administered human NSCs expressing CD and IFN-β migrate into the intracranial tumor bed through the blood vessels, and exhibit antitumor effect by the combined delivery of a suicide gene and a cytotoxic cytokine gene onto the experimental glioma. In a pilot clinical trial conducted in glioma, the IFN-β gene has shown potent antitumor activity in patients with malignant glioma without the adverse systemic effects associated with previous methods of administering IFN-β.

### Human Glioma Cells and NSCs

A human glioma cell line, U251, was obtained from the American Tissue Culture Collection (ATCC, Manassas, Va.) and grown in Eagle's minimal essential medium (Nissui, Tokyo, Japan) containing 10% fetal bovine serum, 5 mM of L-glutamine, 2 mM nonessential amino acids and antibiotics (100 U ml-1 of penicillin and 100 μml-1 of streptomycin) at 37° C. in a humidified atmosphere of 5% CO2. HB1.F3 (F3) human NSC line was generated from the human fetal telencephalon, and was immortalized by transfection with a retroviral vector encoding the v-myc oncogene, as described previously. Neuropathology 2004; 24: 159-171. It has been confirmed that this human NSC line is capable of self renewal and is multipotent, that is, these NSCs can differentiate into cells of the neuronal and glial lineages, both in vivo and in vitro. Id. In this study, the clonal F3.CD.IFN-β line was derived from the parental F3.CD cells. Proc Natl Acad Sci USA 2000; 97: 12846-12851. An expression plasmid was constructed using the pBabePuro retroviral vector (Cell Biolabs, San Diego, Calif.) as the backbone to include the human IFN-β cDNA transcribed from the long terminal repeat ends of the IFN-β gene. Hum Gene Ther 2004; 15: 77-86; J GeneMed 2008; 10: 329-339. The IFN-β.puro plasmid and the MV12 envelope-coding plasmid (provided by Dr K. S. Aboody, Proc Natl Acad Sci USA 2000; 97: 12846-12851) were cotransduced into pA317 cells (ATCC). The supernatant containing the IFN-β-expressing retroviral vector was used for multiple infections of the F3.CD cells.

The transduced F3.CD.IFN-β cells were selected by culturing them for 4 weeks in a medium containing 3 μg ml-1 of puromycin. Successful establishment of the F3.CD.IFN-β cells was confirmed by reverse transcription PCR. The IFN-β transcript in these cells was amplified by touchdown PCR using the following primers:

sense, 50-GCCG CATTGACCATCTATGAGA-30;
antisense, 50-GAGATCT TCAGTTTCGGAGGTAAC-30.

Glyceraldehyde 3-phosphate dehydrogenase was used as a control to confirm equal RNA loading. U251 cells transfected with liposomes containing the human IFN-β gene (pDRSV-IFN-β) were used as positive controls. J GeneMed 2008; 10: 329-339. Parental F3, F3.CD and F3.CD.IFN-β cells were cultured in Dulbecco's modified Eagle's medium with high glucose (Invitrogen, Carlsbad, Calif.) supplemented with 10% fetal bovine serum, fungizone (2.5 μg ml-1) and gentamicin (20 μg ml-1).

### Dil Labeling of NSCs

F3 cells were prelabeled by incubating for 20 min in culture medium containing cell tracker CM-Dil (Invitrogen). CM-Dil has maximum fluorescence emission at 570 nm. Boyden chamber migration assay Filters were coated with a fibronectin (Sigma-Aldrich, St Louis, Mo.; dilution, 1:40 in phosphate-buffered saline (PBS)) solution and air-dried before use. Migration assays were performed in a modified Boyden chamber by using a 24-well HTS FluoroBlok insert system (Falcon Becton Dickinson, Heidelberg, Germany). The inserts contained a polyethylene membrane with a pore size of 8.0 mm, which blocks 99% of the light transmitted at wavelengths 490-700 nm. U251 cells (1×105 cells) were incubated on a 24-well plate.

After 48 h, 1×104 single cells were placed on the top of an insert and incubated for 24 h in Dulbecco's modified Eagle's medium. The cells were fixed using 4% paraformaldehyde in 0.1 M PBS. Then the membrane was cut out of the insert and covered in 4′,6-diamidino-2-phenyl-indole as a mounting medium (absorption at 360 nm and emission at 460 nm; Vectashield, Vector Laboratories, Burlingame, Calif.) between two thin coverslips. The total number of migrated cells at the bottom of the membrane and the number of nonmigrated cells at the top were counted (n=5). The migration index was calculated using the following formula: number of migrated cells at the bottom/number of both migrated and non-migrated cells.

### In Vitro Quantitative Assay for Bystander Killing Effect

The bystander killing effect of F3.CD and F3.CD.IFN-β cells on the U251 cells after 5-FC (Sigma-Aldrich) treatment was quantified. U251 cells were seeded in a 24-well plate (2×104 cells per well) and cocultured with either F3.CD or F3.CD.IFN-β cells at various U251/F3 ratios ranging from 1:0 to 1:4. After 24 hour culture, 5-FC was added to the medium at a final concentration of 500 μg ml-1, and the culture was maintained for 3 more days. Each experiment was performed in triplicate. After the day 3, the cells were rinsed twice with PBS, and the adherent cells were detached using trypsin/EDTA. The number of viable cells was counted by the trypan blue method and expressed as the percentage of untreated U251 cells.

### Clonogenic Assay

The U251 cells were seeded in a 24-well plate (2×104 cells per well) and cocultured with either F3.CD or F3.CD.IFN-β cells at various U251/F3 ratios ranging from 1:0 to 1:4. After 24 hour culture, 5-FC was added to the medium at a final concentration of 500 μg ml-1 and the culture was maintained for 3 more days. The surviving cells were detached from the plates and re-seeded in a sixwell plate at a cell density of 500 cells per well (duplicate). The cells were cultured for 9 days and then stained with 0.25% 1,9-dimethylmethylene blue (Sigma-Aldrich) in 50% ethanol. The number of colonies was counted by two independent observers.

### Intracerebral Glioma Model

The experiments were performed in accordance with the Guidelines for Animal Experiments of the Nagoya University Graduate School of Medicine. BALB/c female nude mice (SLC, Shizuoka, Japan) were anesthetized by administering an intraperitoneal (i.p.) injection of pentobarbital (60-70 mg per kg body weight). The animals were injected with 1×106 U251 cells suspended in 5 μl of PBS using a Hamilton syringe under stereotactic guidance; the injections were administered into the forebrain (2 mm into the lateral side and 1 mm into the anterior side of the bregma; at a depth of 4 mm from the dural surface) for over 5 min. This intracerebral model was reproducible and exhibited a survival of ˜25-40 days.

Histological Study of Intravenously Injected F3 Cells into Intracerebral Glioma

At 14 days after the establishment of the intracerebral glioma model, the animals (n=3) were injected intravenously through the tail vein with 2×106 Dil-labeled F3, F3.CD or F3.CD.IFN-β cells diluted in 100 μl of PBS. The control animals (n=3) were injected with PBS alone. After 7 days, the animals were killed and transcardially perfused with 10% buffered formalin. Paraffin-embedded coronal sections were immunostained with rat antimouse-CD34 antibody (MEC 14.7; HyCult Biotechnology, Uden, Netherlands) followed by anti-rat Alexa Fluor488 (Molecular Probes, Eugene, Oreg.), and nuclei were counterstained with Hoechst 33342. CD34 has been most commonly used in studies of tumor angiogenesis, and the detection of CD34 in endothelial cells can be interpreted as indicative of angiogenesis. The adjacent sections also were processed for hematoxylin and eosin staining.

### Intravenous Transplantation of F3.CD or F3.CD.IFN-β Cells Followed by 5-FC Treatment

At 3 days after the implantation of U251 glioma cells into the brain, the animals were randomly divided, and five mice each were intravenously injected through the tail vein with PBS, F3, F3.CD or F3.CD.IFN-β cells (2×106 cells in 100 μl of PBS). After 2 days, 5-FC was injected intraperitoneally at a dose of 900 mg per kg body weight daily for 10 consecutive days. At 28 days after implantation of glioma cells, the animals were killed and brain sections were processed for hematoxylin and eosin staining. The antitumor effect of the NSCs was evaluated by measuring the long (a) and the short (b) axes of the coronal sections with maximal tumor area. The approximate volume of the tumor (V) was calculated according to the formula

V(mm3)=a×b2/2

The overall survival time from implantation of glioma cells was assessed in another set of mice treated in the same manner.

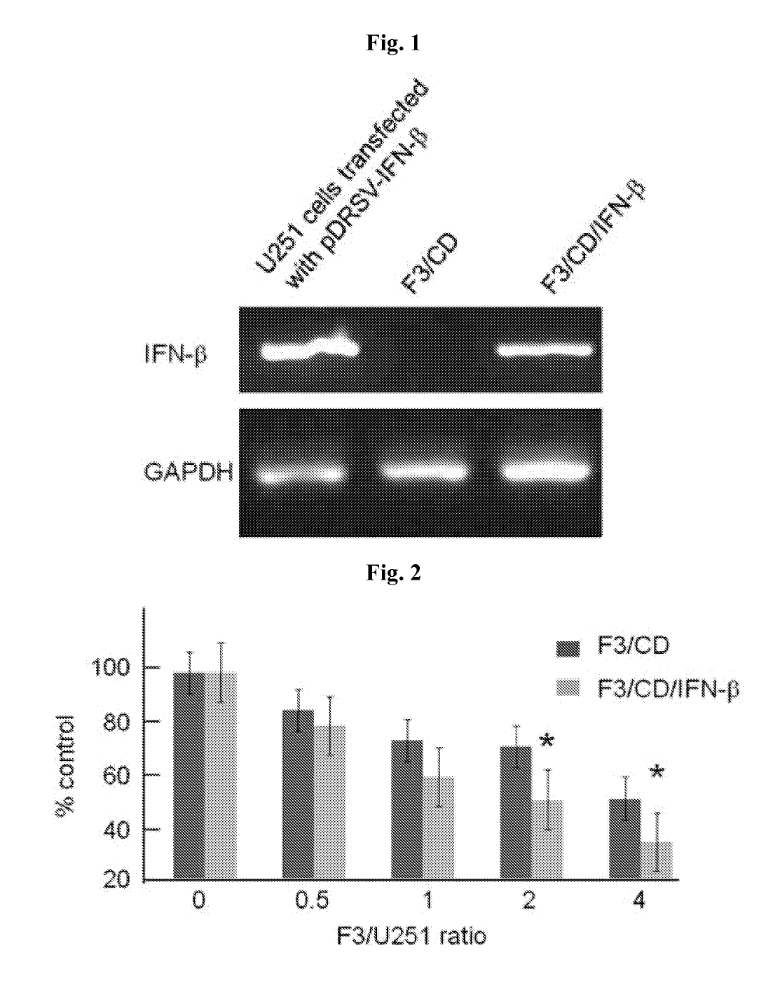

Referring to FIG. 1, it is explained that F3.CD.IFN-β human NSCs produce human IFN-β. FIG. 1 shows the expression of human interferon-β (IFN-β) in the F3.CD/IFN-β cell line. The IFN-β transcript was expressed only in F3.CD.IFN-β human neural stem cells. The expression of the human IFN-β in F3.CD.IFN-β cells was confirmed by reverse transcription PCR. The human IFN-β transcript was found to be expressed in both the clonal cell lines, namely, the F3.CD. IFN-β cell line and the positive control IFN-β-expressing U251 cell line, but not in the parental F3.CD cell line.

### F3.CD.IFN-β Cells Show Higher Bystander Killing Effect on Glioma Cells In Vitro.

To quantify the bystander effect of F3.CD.IFN-β cells on the U251 glioma cells, both types of cells were cocultured at various ratios and subsequently treated with 5-FC. The number of viable cells was assessed after 3 days, and this number was then compared with the number of viable 5-FC-treated U251 cells cultured alone. It was confirmed that the F3.CD and F3.CD. IFN-β cells did not survive after treatment with 500 μg ml-1 5-FC (data not shown).

FIG. 2 shows a bystander killing effect. The U251 cells were seeded in a 24-well plate (2×104 cells per well) and cocultured with either F3.CD or F3.CD.IFN-β cells at various U251/F3 ratios ranging from 1:0 to 1:4. After the day 1 of culture, 5-fluorocytosine (5-FC) was added to the medium at a final concentration of 500 μg ml-1, and the culture was maintained for 3 more days. Each experiment was performed in triplicate. The number of viable cells was counted by the trypan blue method, and expressed as the percentage of untreated U251 cells. The number of viable cells decreased with an increase in the F3/U251 ratio in both the cocultures, that is, in F3.CD+U251 and F3.CD.IFN-β+U251. Notably, the bystander killing effect exhibited by the F3.CD.IFN-b cells was more significant than that of the F3.CD cells, at the F3/U251 ratio of 2:1 and 4:1 (*P<0.05).

As shown in FIG. 2, the number of viable cells decreased with an increase in the F3/U251 ratio in both groups, that is, F3.CD+U251 and F3.CD.IFN-β+U251. Notably, the bystander killing effect exhibited by the F3.CD.IFN-β cells was more significant than that of the F3.CD cells, at the F3/U251 ratio of 2:1 and 4:1 (P<0.05). Consistent with this result, the clonogenic potential of the U251 cells surviving after coculture with F3.CD.IFN-β and 5-FC treatment was significantly lower than the clonogenic potential for similar U251 cells that were cocultured with F3.CD cells.

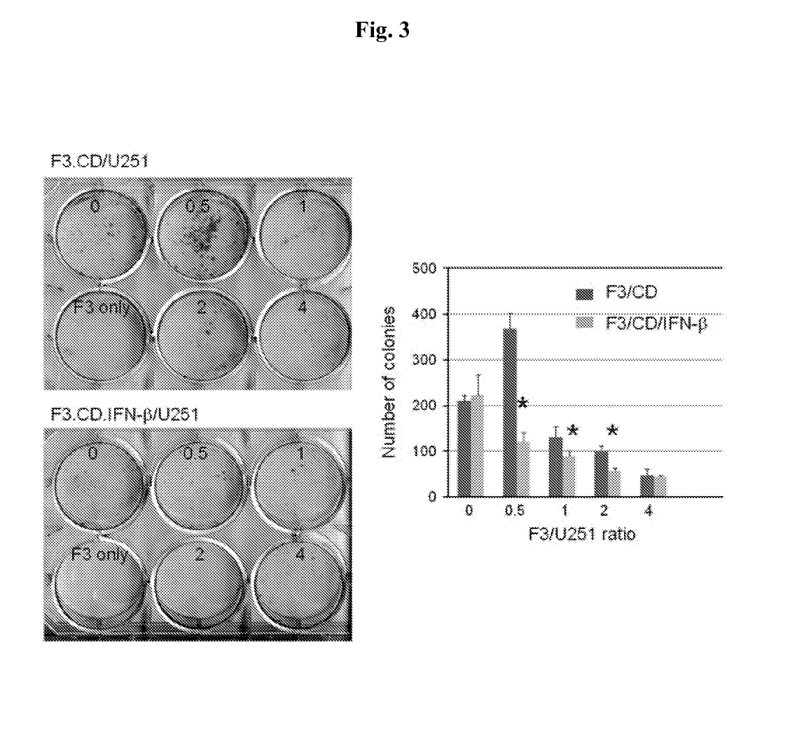

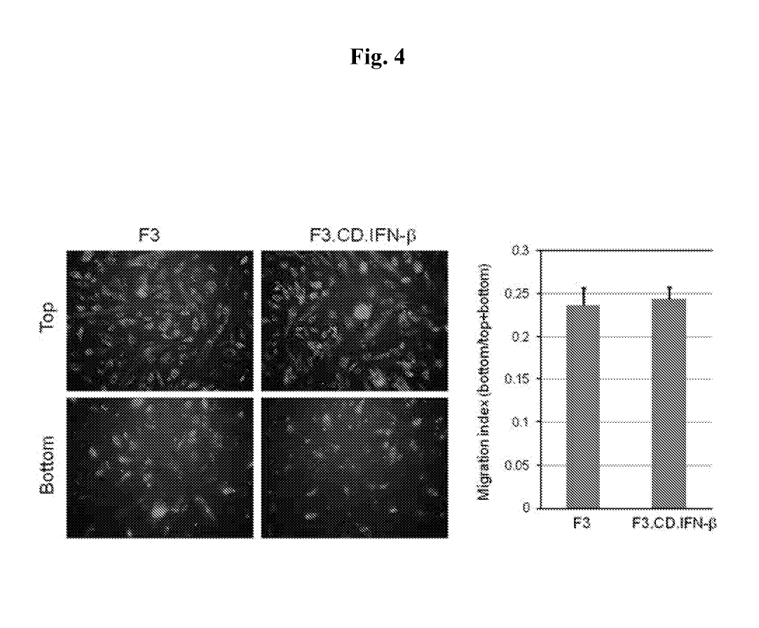

FIG. 3 shows the clonogenic assay. U251 cells were seeded in a 24-well plate (2×104 cells per well) and cocultured with either F3.CD or F3.CD.IFN-β cells at various U251/F3 ratios ranging from 1:0 to 1:4. After a 24 hour culture, 5-fluorocytosine (5-FC) was added to the medium at a final concentration of 500 μg ml-1, and the culture was maintained for 3 more days. The surviving cells were detached from the plates and re-seeded in a six-well plate at a cell density of 500 cells per well (duplicate). The cells were cultured for 9 days and then stained with 0.25% 1,9-dimethylmethylene blue (Sigma-Aldrich) in 50% Ethanol. The number of colonies was counted by two independent observers. The clonogenic potential of surviving U251 cells after coculture with F3.CD.IFN-b after 5-FC treatment was significantly reduced compared with coculture with F3.CD (*P<0.05).

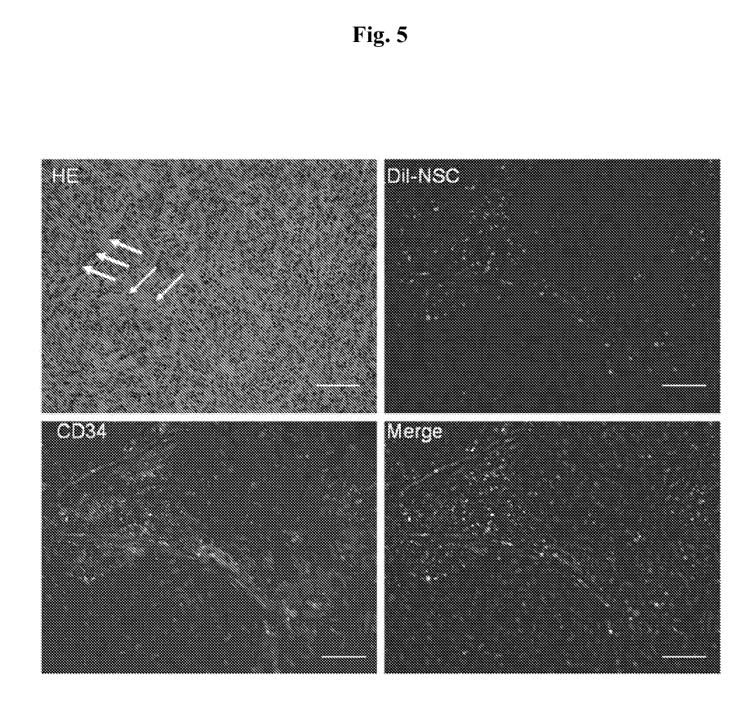

Referring to FIG. 4, it is explained that F3 cells migrate to intracranial glioma in mice. The in vitro migration assay revealed that F3.CD. IFN-β cells had the same migratory pattern as that of the parental F3 cells. FIG. 4 shows the migration assay in vitro: U251 cells (1×105) were plated on the 24-well plate and cultured for 48 hours. CM-Dil-labeled F3 or F3.CD.IFN-β cells (2×104) were seeded into the upper wells of the FluoroBlok inserts. After a 24 hour incubation, migrated (bottom) and non-migrated cells (top) were counted. There was no significant difference between the migration indices of these two cell lines. Next, to ascertain the migratory capability of the NSCs from the vessels to the tumor mass, the Dil-labeled F3.CD.IFN-β cells were injected into the tail vein, and the newly formed tumor vessels were immunostained by using an anti-CD34 antibody. The Dil-labeled NSCs were found to be presenting both, tumor stroma and tumor parenchyma. Thus, NSCs appeared to migrate into the tumor parenchyma extending from tumor vessels as shown in FIG. 5. FIG. 5 shows the migration of F3.CD.IFN-β cells into the tumor parenchyma. To ascertain the migratory capability of the neural stem cells (NSCs) from the vessels to the tumor parenchyma, the Dil-labeled F3.CD.IFN-β cells (red) were injected into the tail vein, and the newly formed tumor vessels were immunostained by using an anti-CD34 antibody (green). The Dil-labeled NSCs were found to be present in both, tumor stroma and tumor parenchyma. An adjacent section was also stained with hematoxylin and eosin, and the field relevant to the fluorescence images is displayed. The arrows indicate tumor vessels. (Scale bar, 100 μm.)

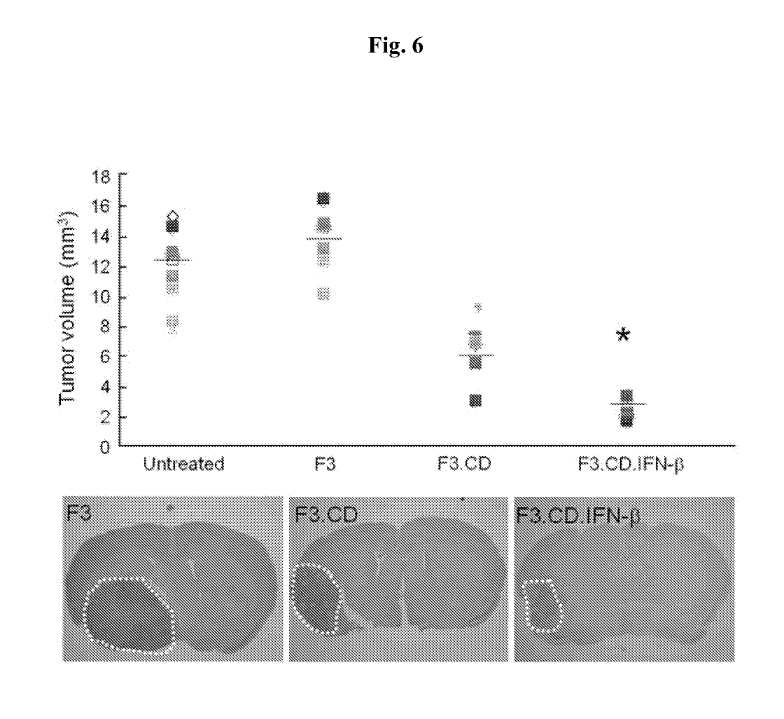

Referring to FIG. 6, it is explained that F3.CD. IFN-β cells reduce tumor burden in experimental glioma in mice. The growth inhibitory effect of genetically engineered NSCs (for example, F3.CD.IFN-β and F3.CD cells) on glioma cells in vivo was also observed. The mice with the U251-derived intracerebral tumor were injected with F3 cells, and the i.p. injections of 5-FC were administered for the next 10 days. The volume of the tumor was assessed on day 28 after FC treatment. As shown in FIG. 6, the residual tumor mass obtained in the group treated with F3.CD. IFN-β was much smaller than that obtained in the group treated with F3.CD cells (*P<0.05).

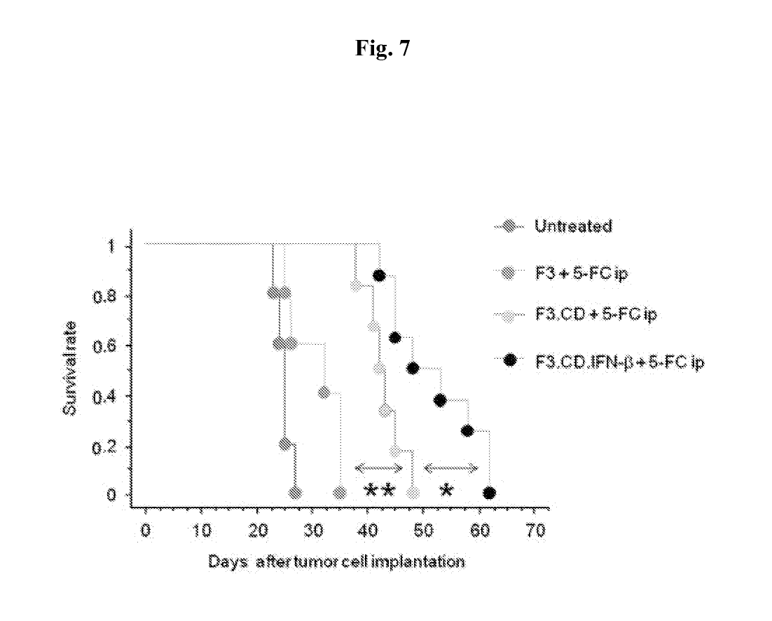

Referring to FIG. 7, it is explained that F3.CD. IFN-β cells increase the survival periods in experimental animals. FIG. 7 shows the survival time of experimental animals. Mice were inoculated with U251 intracranially, and subsequently with each type of neural stem cells (NSCs) (F3.CD.IFN-β (n=8), F3.CD (n=6) and F3 (n=5)), followed by intraperitoneal injection of prodrug 5-fluorocytosine (5-FC) and compared with untreated animals (n=5). The survival time from U251 cell inoculation was measured. The rates of survival of mice treated with F3.CD.IFN-β cells were significantly higher than those of mice treated with F3.CD (*P<0.05); mice that received F3.CD therapy had significantly higher survival rates than those that received only F3 cells (**P<0.005).

To determine whether F3.CD.IFN-β implantation followed by i.p. injections of 5-FC can produce therapeutic benefits in vivo, the implantation was performed 3 days after intracranial inoculation of U251 cells. Survival periods of mice first inoculated with U251 intracranially, and subsequently with each type of NSCs (F3.CD.IFN-b, F3.CD and F3) and i.p. injected with 5-FC were measured. The survival of mice treated with F3.CD. IFN-β was significantly longer than that of mice treated with F3.CD or F3 cells. These results suggest that the antitumor effect of F3.CD.IFN-β cells on glioma cells is greater than the antitumor effect of the F3.CD cells.

In the present invention the genetically engineered human NSCs expressing CD and IFN-β appear to exert an additive effect in destroying intracerebral gliomas. The number of viable tumor cells present in a coculture of 5-FC-treated F3.CD.IFNβ+U251 cells was ˜60% of that in a co-culture of 5-FC-treated F3.CD+U251 cells at a F3/U251 ratio of 2:1 or 4:1 as shown in FIG. 2. Further, compared with the mice injected with F3.CD, the mice intravenously injected with F3.CD.IFN-β showed a significantly higher reduction in the tumor volume as well as a longer survival period as shown in FIGS. 6 and 7.

Interferon-β is a type I IFN that exerts pleiotropic biological effects. Semin Cancer Biol 2000; 10: 125-144. Previous preclinical and experimental studies have investigated the application of cationic liposomes for delivering the IFN-β gene in glioma patients. In vitro experiments showed that the cationic liposome-mediated human IFN-β gene transfer into the cultured human glioma cells induced a cytocidal but not a cytostatic response even in IFN-resistant human glioma cell lines, probably by inducing apoptosis. Semin Cancer Biol 2000; 10: 125-144. Cationic liposome-mediated IFN-β gene transfer exhibited a much stronger inhibition of glioma cell growth than exogenous IFN-β. A 40-fold increase in the concentration of IFN-β was required to obtain an inhibitory effect similar to that observed with exogenous transfer of the IFN-β gene.

The invention is directed, for example, at facilitating a sustained higher expression of IFN-β in the microenvironment in order to induce a direct apoptotic effect on the surrounding tumor cells. In vivo experiments using mice implanted with human glioma cells revealed that local administration of cationic liposomes containing the human IFN-β gene induced an apparent reduction in the tumor growth and prolonged the survival. Cancer Immunol Immunother 1998; 47: 227-232; Gene Ther 1999; 6: 1626-1633; J Neurooncol 2000; 47:117-124; Biochem Mol Biol Int 1994; 32: 167-171.

On the basis of these observations, a phase I clinical trial of IFN-β gene therapy was performed on five patients with recurrent malignant glioma. Hum Gene Ther 2004; 15: 77-86. At 10 weeks after treatment initiation, two patients showed more than 50% tumor reduction, whereas others did not show any significant improvement. The median survival was longer in the treated subjects than in the matched historical controls from our institution. After gene therapy, significant changes were observed in the histology and gene expression related to immune response, apoptosis and neovascularization. J Gene Med 2008; 10: 329-339. A recent study has reported the findings of a phase I clinical trial, in which stereotactic injections of IFN-β expressing adenoviral vectors were administered to 11 patients with malignant glioma and resulted in modest clinical outcome. Mol Ther 2008; 16: 618-626. However, local administration of therapeutic IFN-β vectors used in these clinical trials could not address the issues of selective targeting of infiltrative satellite tumors. To overcome this limitation, the inherently migratory, tumor-tropic NSCs can serve as a potentially powerful therapeutic tool. NSCs display remarkable tropism and migratory capacity to sites of malignant growth. Neoplasia 2005; 7: 623-629; Stem Cells 2008; 26: 1575-1586. The invention utilizes the human F3 NSC cell line, as it is a well-characterized and a well-established human NSC line. Proc Natl Acad Sci USA 2000; 97: 12846-12851; Gene Ther 2007; 14: 1132-1142; Mol Ther 2009; 17: 570-575; Clin Cancer Res 2006; 12: 5550-5556; Neuropathology 2004; 24: 159-171.

It is important that this F3 line was used for a number of reasons. First, no signs of local or systemic toxicity were observed in case of animals injected with F3 cells alone. Furthermore, these cells can be modified to stably express a therapeutic transgene. In NSC-based gene therapy strategies targeting brain tumors, NSCs were mostly used to transport the CD/5-FC prodrug system to the tumor cells. Proc Natl Acad Sci USA 2000; 97: 12846-12851; Cancer Gene Ther 2003; 10: 396-402; Gene Ther 2007; 14: 1132-1142; Mol Ther 2009; 17: 570-575; Clin Cancer Res 2006; 12: 5550-5556; Neuro Oncol 2006; 8: 119-126. In mice, F3 human NSCs transiently expressing human IFN-β display tropism for sites of disseminated neuroblastoma, resulting in significant tumor growth. J Pediatr Surg 2007; 42: 48-53. The sustained expression of IFN-β at disseminated sites of microscopic disease represents a novel therapeutic approach. The present invention implements additive efficacy of NSCs for delivering CD as well as IFN-β to the tumor site.

Further studies are required to elucidate the mechanisms by which IFN-β intensifies the bystander effect of CD against glioma cells. Moreover, the application of NSCs in clinical settings raises some concerns. A recent study reported an NSC-derived brain tumor in a patient with ataxia telangiectasia who had been administered intracerebellar and intrathecal injections of human fetal NSCs. PLoS Med 2009; 6: e1000029. In this study, we systematically delivered a human NSC cell line immortalized by v-myc. In our previous publication, we have reported that intravenously injected NSCs tend to be trapped in the spleen, kidney and liver. Neurosci Lett 2007; 426: 69-74. Systemic administration of immortalized NSCs has the potential to cause neoplasm formation. These issues need to be addressed before clinical application. Nevertheless, our study indicates that the toxic effect against glioma cells exerted by the invention's combination of two treatments is more effective than that exerted by CD-based suicide strategy. These findings support the possible application of a one-two-punch combination therapy for the treatment of malignant gliomas.

While the invention has been described with reference to the exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method of treating a tumor in an individual in need thereof, the method comprising:
a) providing human stem cells, wherein the stem cells are modified to deliver or express substances that have direct antitumor effect, and
b) systemically delivering the stem cells to the individual.

2. The method of claim 1, wherein the stem cells are neural stem cells (NSCs).

3. The method of claim 1, wherein the tumor is a nervous system tumor, brain or spinal metastasis from an extracranial tumor, or an extracranial tumor with metastasis to non-neural structures.

4. The method of claim 1, wherein the systemic delivery is performed using intravascular, or intra-cerebrospinal fluid (CSF) injection.

5. The method of claim 1, wherein the substance are genes that confer therapeutic benefit.

6. The method of claim 5, wherein the genes encode a suicide gene cytosine deaminase (carboxylesterase or herpes simplex-1 thymidine kinase) and a cytokine interferon-beta (s-TRAIL, IL-4 or IL-12).

7. The method of treating a tumor in an individual in need thereof, the method comprising:
a) providing a preparation containing neural stem cells that express cytosine deaminase and interferon-beta;
b) systemically delivering said neural stem cells to the individual;
c) the interferon-beta- and cytosine deaminase-encoding cells produce therapeutic interferon-beta; and
d) administering 5-fluorocytosine, wherein the interferon-beta- and cytosine deaminase-encoding cells convert the non-toxic 5-fluorocytosine to toxic and therapeutic 5 fluorouracil.

8. The method of claim 7, wherein the tumor is a nervous system tumor, brain or spinal metastasis from an extracranial tumor, or an extracranial tumor with metastasis to non-neural structures.

9. The method of claim 7, wherein said systemic delivery is intravascular or intra-CSF injection.

10. A pharmaceutical preparation comprising liquid nitrogen-frozen stem cells or actively growing stem cells in a pharmaceutically acceptable diluent.

11. A kit for system treatment of tumor, the kit comprising a vial of frozen stem cells or actively growing stem cells, said cells have been engineered to exert therapeutic effects on the tumor and being capable of migrating to the tumor, a container of a pharmaceutical grade solution for suspending the stem cells and/or catheter.


SEQUENCE LISTING (2 sequences)

SEQ ID NO: 1 - DNA, 22 bp, Artificial sequence
  Human interferon-Beta PCR primer
gccgcattgaccatctatgaga

SEQ ID NO: 2 - DNA, 24 bp, Artificial Sequence
  Human interferon-Beta PCR primer
gagatcttcagtttcggaggtaac